(19) United States
(12) Patent Application Publication
(10) Pub. No.: US 2009/0181319 A1
(43) Pub. Date: Jul. 16, 2009

(54) AROMATIC FLUORINE-FREE PHOTOACID GENERATORS AND PHOTORESIST COMPOSITIONS CONTAINING THE SAME

(75) Inventors: Wenjie Li, Poughkeepsie, NY (US); Sen Liu, Highland Park, NJ (US); Wu-Song Huang, Brewster, NY (US); Pushkara R. Varanasi, Poughkeepsie, NY (US); Irene Popova, Beacon, NY (US)

Correspondence Address: INTERNATIONAL BUSINESS MACHINES CORPORATION;DEPT. 18G, BLDG. 321-482, 2070 ROUTE 52, HOPEWELL JUNCTION, NY, 12533, US

(73) Assignee: INTERNATIONAL BUSINESS MACHINES CORPORATION, Armonk, NY (US)

(21) Appl. No.: 12/015,041

(22) Filed: Jan. 16, 2008

(51) Int. Cl. G03F 7/004; G03F 7/26; C07C 317/14

(52) U.S. Cl. 430/281.1; 430/322; 568/30; 568/35; 568/28

(57) ABSTRACT

Fluorine-free photoacid generators and photoresist compositions containing fluorine-free photoacid generators are enabled as alternatives to PFOS/PFAS photoacid generator-containing photoresists. The photoacid generators are characterized by the presence of a fluorine-free aromatic sulfonate anionic component having one or more electron withdrawing groups. The photoacid generators preferably contain a fluorine-free onium cationic component, more preferably a sulfonium cationic component. The photoresist compositions preferably contain an acid sensitive imaging polymer having a lactone functionality. The compositions are especially useful for forming material patterns using 193 nm (ArF) imaging radiation.

### BACKGROUND OF THE INVENTION

In the field of semiconductor manufacturing, optical lithography has been the mainstream approach used in patterning semiconductor devices. In typical prior art lithography processes, UV light is projected onto a silicon wafer coated with a thin layer of photosensitive resist (photoresist) through a mask that defines a particular circuitry pattern. Exposure to UV light, followed by subsequent baking, induces a photochemical reaction which changes the solubility of the exposed regions of the photoresist. Thereafter, an appropriate developer, usually an aqueous base solution, is used to selectively remove photoresist either in the exposed regions (positive-tone photoresists) or, in the unexposed regions (negative-tone photoresists). The pattern thus defined is then imprinted on the silicon wafer by etching away the regions that are not protected by the photoresist with a dry or wet etch process.

One type of photoresist employed in the prior art is a chemically amplified photoresist which uses acid catalysis. A typical prior art chemically amplified photoresist, for example, is formulated by dissolving an acid sensitive polymer and a photoacid generator in a casting solution. A chemically amplified photoresist is especially useful when relatively short wavelength radiation is employed, including deep UV radiation 150-315 nm wavelengths, and mid-UV radiation, e.g., 350-450 nm wavelengths. The shorter wavelengths are typically desired to increase resolution, and thus, decrease feature size of the semiconductor devices, but fewer photons are radiated for a given energy dose.

Accordingly, higher exposure doses are typically required when using UV radiation to obtain a sufficient photochemical response in the photoresist unless a chemically amplified photoresist is employed. In a chemically amplified photoresist, acid sensitivity of the base polymer exists because acid sensitive side chain groups are bonded to the polymer backbone. When such a photoresist is exposed to radiation, the photoacid generator produces an acid that, when the photoresist is heated, causes catalytic cleavage of the acid sensitive side chain groups. A single acid catalyst molecule generated in this manner may be capable of cleaving multiple side chain groups, thus allowing lower exposure doses for the needed photochemical response.

Because of the relatively low intensity of ArF laser source and relatively high binding energy of acid labile moieties in ArF photoresist, photoacid generators which can produce stronger Bronsted acid with much higher sensitivity are preferred to realize such chemical amplification in commercial lithography. Fluorine-containing onium salts, such as perfluoronated octyl sulfonate (PFOS) and perfluoronated alkyl sulfonate (PFAS), are generally preferred used as the photoacid generator in ArF photoresist system in part because they result in generation of strong acid.

In recent years, there has been a desire in the microelectronics industry to eliminate the use of perfluorinated carbons (PFCs) such as PFOS and PFAS. Thus, there is a desire to find alternative photoacid generators which can be used without adversely impacting the performance of lithographic processes. There has also been a desire to minimize or eliminate fluorine content in photoresist in order to improve etch resistance and to improve process latitude in high numeric aperture (NA>0.95) imaging processes.

Some attempts have been made to develop photoresist formulations that do not use perfluorinated carbon-containing photoacid generators, however these have largely been unsuccessful in achieving performance comparable to formulations using PFOS. Thus, there is a need for new and improved photoacid generators and chemically amplified photoresist compositions that enable the substantial reduction or avoidance of PFCs and/or fluorine in photoresist compositions.

### SUMMARY OF THE INVENTION

The invention provides fluorine-free photoacid generators and photoresist compositions comprising fluorine-free photoacid generators. The photoacid generators of the invention provide a viable alternative to the PFC-containing photoacid generators currently used in the industry. The invention also provides photoresist compositions containing fluorine-free photoacid generators that show excellent optical clarity, thermal stability and lithographic performance (photospeed comparable with photoresists using of the commercial PFC-containing photoacid generators).

In one aspect, the invention encompasses fluorine-free photoacid generators having a sulfonium cationic component and an anionic component having the structure:

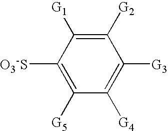

wherein each of G₁-G₅ is fluorine-free and at least one of G₁-G₅ is an electron withdrawing moiety. Preferably, the anionic component is fluorine-free. Preferably, at least one of G₁-G₅ is an electron withdrawing moiety selected from the group consisting of CN, NO, NO₂, Cl, Br, I, SO₂Me, CHO. The other G₁-G₅ moieties are preferably selected independently from the group consisting of H; linear, branched, tertiary, or cyclic alkyl; linear, branched, tertiary or cyclic alkoxyl; unsubstituted and substituted phenyl; unsubstituted and substituted naphthyl; or unsubstituted and substituted fluorenyl.

In another aspect, the invention encompasses a chemically amplified photoresist composition comprising:
(a) an acid sensitive polymer having at least one lactone moiety, and
(b) a photoacid generator comprising an anionic component having the structure

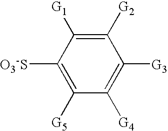

wherein each of G₁-G₅ is fluorine-free and at least one of G₁-G₅ is an electron withdrawing group. Preferably, at least one of G₁-G₅ is a electron withdrawing moiety selected from the group consisting of CN, NO, NO₂, Cl, Br, I, SO₂Me, CHO. The other G₁-G₅ moieties are preferably selected independently from the group consisting of H; linear, branched, tertiary, or cyclic alkyl; linear, branched, tertiary or cyclic alkoxyl; unsubstituted and substituted phenyl; unsubstituted and substituted naphthyl; or unsubstituted and substituted fluorenyl. The photoacid generator preferably includes an onium cationic component, more preferably a sulfonium cationic component.

In another aspect, the invention encompasses photoresist compositions containing a photoresist polymer component and a fluorine-free photoacid generator having a sulfonium cationic component and an anionic component having the structure:

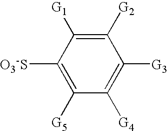

wherein each of G₁-G₅ is fluorine-free and at least one of G₁-G₅ is an electron withdrawing group. Preferably, at least one of G₁-G₅ is a moiety selected from the group consisting of CN, NO, NO₂, Cl, Br, I, SO₂Me, CHO. The other G₁-G₅ moieties are preferably selected independently from the group consisting of H; linear, branched, tertiary, or cyclic alkyl; linear, branched, tertiary or cyclic alkoxyl; unsubstituted and substituted phenyl; unsubstituted and substituted naphthyl; or unsubstituted and substituted fluorenyl.

The invention also encompasses processes for using the compositions of the invention to form patterned material structures on a substrate. These methods preferably involve the use of 193 nm (ArF) imaging radiation.

These and other aspects of the invention are described in further detail below.

### DETAILED DESCRIPTION OF THE INVENTION

The invention provides fluorine-free photoacid generators. The photoacid generators of the invention provide a viable alternative to the fluorine-containing photoacid generators currently used in the industry. The invention also provides photoresist compositions containing fluorine-free photoacid generators that show excellent optical clarity, thermal stability and lithographic performance (photospeed comparable with photoresists using of the commercial PFC-containing photoacid generators).

In one aspect, the photoacid generators of the invention are characterized by the presence of a sulfonium cationic component (with certain photoresists, other onium cationic components may be used). The photoacid generators of the invention are not limited to any specific sulfonium cation. Preferred sulfonium cation structures contain aromatic moieties in one or more pendant groups R, R′, or R″:

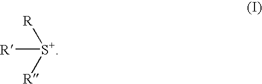

More preferred sulfonium cation structures are:

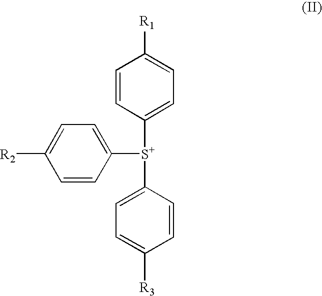

where R₁, R₂, or R₃ is each independently selected from the group consisting of H; linear, branched, tertiary, or cyclic alkyl (preferably C₁-C₁₂ or C₄-C₁₂ in the case or tertiary and cyclic); linear, branched, tertiary or cyclic alkoxyl (preferably C₁-C₁₂ or C₄-C₁₂ in the case or tertiary and cyclic); unsubstituted and substituted phenyl; unsubstituted and substituted naphthyl; or unsubstituted and substituted fluorenyl. Another preferred sulfonium cation has a structure:

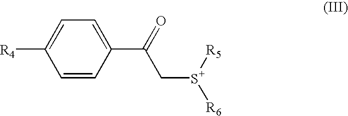

where R₄ is selected from the group consisting of each R is independently selected from the group consisting of H; linear, branched, tertiary, or cyclic alkyl (preferably C₁-C₁₂ or C₄-C₁₂ in the case or tertiary and cyclic); linear, branched, tertiary or cyclic alkoxyl (preferably C₁-C₁₂ or C₄-C₁₂ in the case or tertiary and cyclic); unsubstituted and substituted phenyl; unsubstituted and substituted naphthyl; or unsubstituted and substituted fluorenyl. R₅ and R₆ are each selected from the group consisting of each R is independently selected from the group consisting of H; linear, branched, tertiary, or cyclic alkyl (preferably C₁-C₁₂ or C₄-C₁₂ in the case or tertiary and cyclic); linear, branched, tertiary or cyclic alkoxyl (preferably C₁-C₁₂ or C₄-C₁₂ in the case or tertiary and cyclic); unsubstituted and substituted phenyl; unsubstituted and substituted naphthyl; or unsubstituted and substituted fluorenyl, or R₅ and R₆ are collectively an alkylene (CH₂)ₙ (preferably C₂-C₁₂) chain.

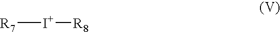

where R₇ and R₈ are each selected from the group consisting of each R is independently selected from the group consisting of H; linear, branched, tertiary, or cyclic alkyl (preferably C₁-C₁₂ or C₄-C₁₂ in the case or tertiary and cyclic); linear, branched, tertiary or cyclic alkoxyl (preferably C₁-C₁₂ or C₄-C₁₂ in the case or tertiary and cyclic); unsubstituted and substituted phenyl; unsubstituted and substituted naphthyl; or unsubstituted and substituted fluorenyl.

The anionic component of the photoacid generators of the invention has the structure:

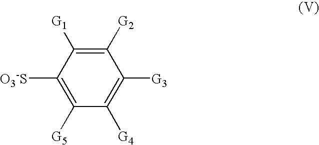

where at least one of G₁-G₅ is an electron withdrawing moiety. Preferably, at least one of G₁-G₅ is an electron withdrawing moiety independently selected from the group consisting of CN, NO, NO₂, Cl, Br, I, SO₂Me, CHO. Preferably, one or two of the Gₓ moieties are electron withdrawing groups. The electron withdrawing groups may be the same or different in each anionic component. The other G₁-G₅ moieties are preferably selected independently from the group consisting of H; linear, branched, tertiary, or cyclic alkyl (preferably C₁-C₁₂ or C₄-C₁₂ in the case or tertiary and cyclic); linear, branched, tertiary or cyclic alkoxyl (preferably C₁-C₁₂ or C₄-C₁₂ in the case or tertiary and cyclic); unsubstituted and substituted phenyl; unsubstituted and substituted naphthyl; or unsubstituted and substituted fluorenyl. Examples of anionic components are:

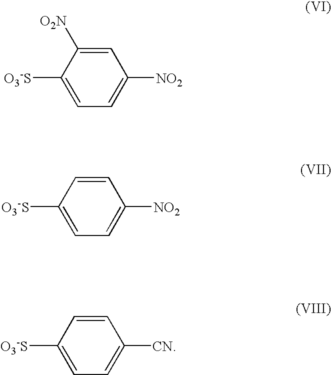

The invention is not limited to any specific method for synthesizing the photoacid generators of the invention. One possible synthesis route is shown in Scheme 1 below. In this scheme, photoacid generators of the invention can be made in a one-pot reaction from sulfonyl chloride to its corresponding silver sulfonate without the intermediate sulfuric acid, which is otherwise detrimental to the cyano group. Cyano-substituted phenyl sulfonyl chloride can be reacted with silver carbonate to afford the silver salt in solid phase at almost quantitative yield. The resulting silver salt can then reacted with corresponding sulfonium or iodonium cation source to afford the desirable photoacid generators. The chemical structures of the resulting compounds were confirmed by their ¹H NMR spectra. The detailed syntheses of more photoacid generators are shown in Scheme 2.

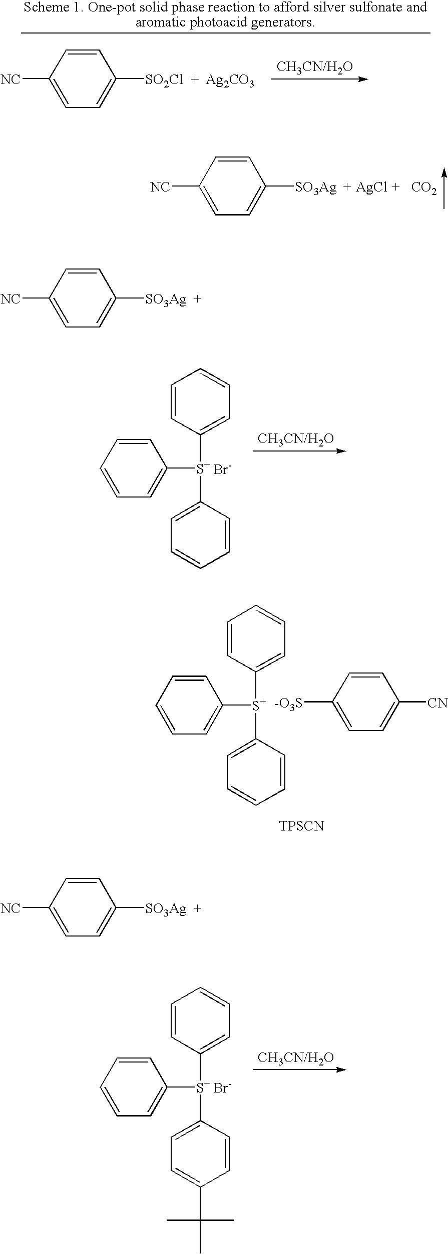

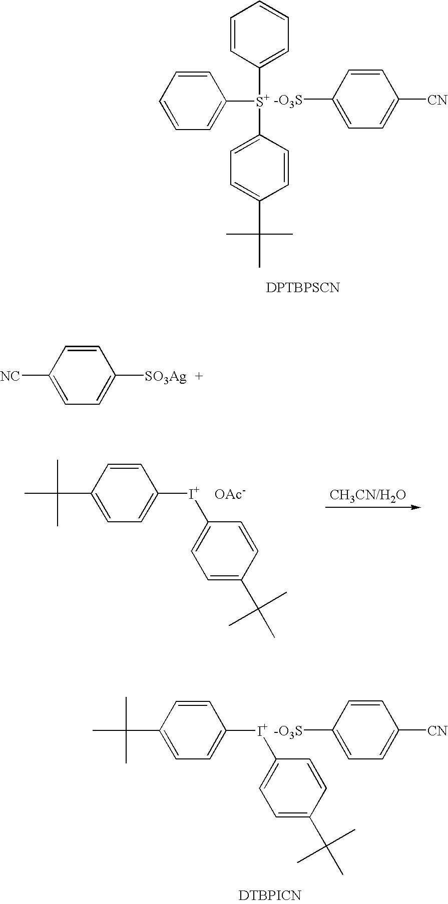

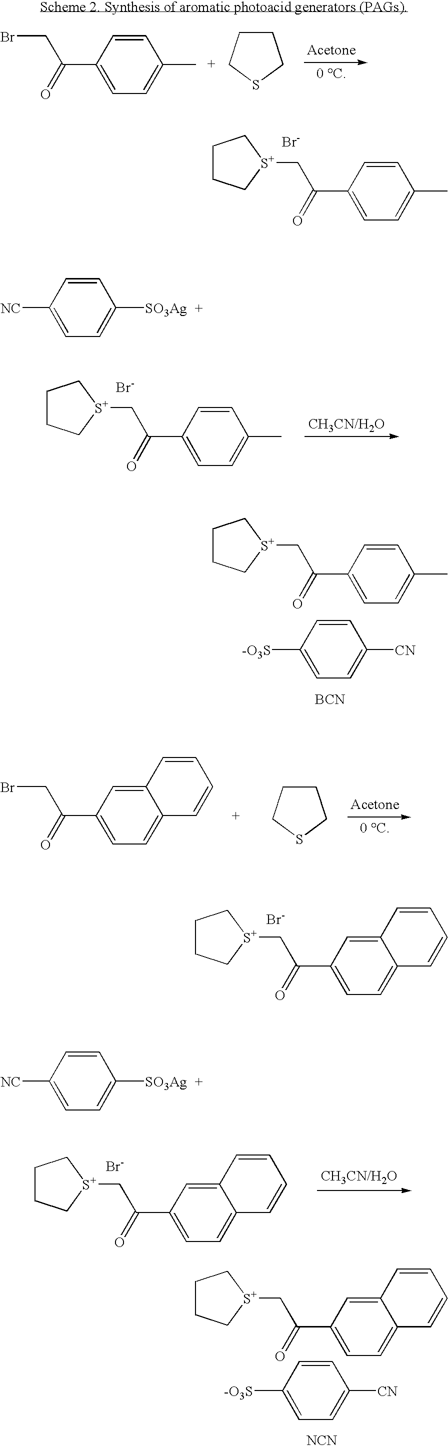

The invention encompasses a chemically amplified photoresist composition containing fluorine-free photoacid generators. The photoresist compositions comprise an acid sensitive imaging polymer and a fluorine-free acid generator. The imaging polymer is preferably capable of undergoing chemical transformations upon exposure of the photoresist composition to UV light whereby a differential in the solubility of the polymer in either the exposed regions or the unexposed regions is created. That is, the base polymers employed in the present invention include any acid sensitive polymer having acid sensitive side chains which can undergo catalytic cleavage in the presence of an acid generated by the inventive photoacid generator. The imaging polymer may be either a positive-tone imaging polymer or a negative-tone imaging polymer. In such polymers, the acid sensitivity exists because of the presence of acid sensitive side chains that are bonded to the polymer backbone. Such acid sensitive polymers including acid sensitive side chains are conventional and are well known in the art. Preferably, the imaging polymer is one suitable for use in 193 nm (ArF) lithography.

In some embodiments, the acid sensitive side chains of the acid sensitive polymers are protected with various acid labile protecting groups that are well known to those skilled in the art. For example, the acid sensitive side chains may be protected with high activation energy protecting groups such as t-butyl ester or t-butyl carbonyl groups, a low activation energy protecting group such as acetal, ketal, or silyethers, or a combination of both low and high activation energy protecting groups may also be used. Most preferably, the imaging polymer of the invention contains a lactone moiety, more preferably a pendant lactone moiety. Examples of imaging polymers containing lactone moieties are well known in the art. See for example US Published Patent Application No. 20060216643A1, and U.S. Pat. Nos. 7,087,356, 7,063,931, 6,902,874, 6,730,452, 6,627,391, 6,635,401 and 6,756,180. Some preferred lactone-containing monomeric units for inclusion in the imaging polymer are:

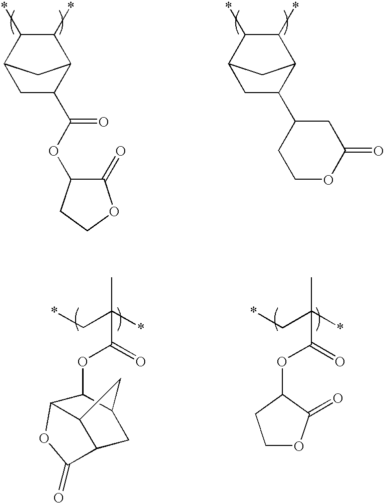

Preferred imaging polymers contain at least about 5 mole % of lactone-containing monomeric units based on the total monomeric units in the imaging polymer, more preferably about 10-50 mole %, most preferably 15-35 mole %.

The photoresist compositions of the invention preferably contain a solvent which is capable of dissolving the acid sensitive polymer. Illustrative examples of such solvents include, but are not limited to: ethers, glycol ethers, aromatic hydrocarbons, ketones, esters and the like. A solvent system including a mixture of the aforementioned solvents is also contemplated herein. Suitable glycol ethers include: 2-methoxyethyl ether (diglyme), ethylene glycol monomethyl ether, propylene glycol monomethyl ether, propylene glycol monomethylether acetate (PGMEA) and the like. Examples of suitable aromatic hydrocarbon solvents that include: toluene, xylene, and benzene. Examples of ketones include: methylisobutylketone, 2-heptanone, cycloheptanone, and cyclohexanone. An example of an ether solvent is tetrahydrofuran, whereas ethyl lactate and ethoxy ethyl propionate are examples of ester solvents that may be employed herein. PGMEA is a preferred solvent.

In addition to the above components, the photoresist composition may also include other components such as photosensitizers, bases, surfactants or other additives. If desired, combinations or mixtures of these other components may be used (e.g., a photosensitizer and a base).

The optional photosensitizer is preferably one containing chromophores that are capable of absorbing irradiation in 193 nm (ArF) lithography. Illustrative examples of such compounds include, but are not limited to: 9-anthracene methanol, coumarins, 9,10-bis(trimethoxysily ethynyl) anthracene and polymers containing these chromophores. Of these compounds, it is highly preferred to use 9-anthracene methanol as the photosensitizer.

The optional bases that can be employed in the present invention include, but are not limited to: berberine, cetyltrimethylammonium hydroxide, 1,8-bis(dimethylamino)naphthalene, tetrabutyl ammonium hydroxide (TBAH), amines, polymeric amines and the like. When a base is employed with the inventive chemically amplified photoresist composition, it is highly preferred to use TBAH as the base.

The optional surfactants that can be employed in the photoresist compositions include any surfactant that is capable of improving the coating homogeneity of the chemically amplified photoresist composition of the present invention. Illustrative examples include: fluorine-containing surfactants such as 3M's FC-430® and siloxane-containing surfactants such as Union Carbide's Silwet® series.

The photoresist compositions of the invention preferably comprise from about 5 to about 30 weight % imaging polymer, from about 50 to about 94.9 weight % solvent, and from about 0.1 to about 20 weight % fluorine-free photoacid generator (the weight % fluorine-free photoacid generator being based on the total weight of imaging polymer present in the composition). When a photosensitizer is employed, it is preferably present in an amount of from about 0.001 to about 8 weight %, based on the total weight of imaging polymer. If a base is employed, the optional base is preferably present in an amount of from about 0.1 to about 1 weight %, based on the total weight of imaging polymer. When a surfactant is employed, it is preferably present in amount of from about 0.001 to about 0.1 weight %, based on the total weight of imaging polymer.

More preferably, the photoresist composition comprises from about 10 to about 20 weight % of imaging polymer, from about 80 to about 90 weight % solvent, and from about 1 to about 5 weight % of fluorine-free photoacid generator (based on the total weight of imaging polymer present in the composition) optionally, from about 0.01 to about 5 weight % photosensitizer, based on the total weight of imaging polymer, optionally, from about 0.1 to about 0.5 weight % base, based on the total weight of imaging polymer, and optionally, from about 0.001 to about 0.01 weight % surfactant, based on the total weight of imaging polymer.

Note that the amounts given above are exemplary and that other amounts of each of the above components, which are typically employed in the photolithography industry, can also be employed herein.

The invention also encompasses processes for using the compositions of the invention to form patterned material features on a substrate. These methods preferably involve the use of 193 nm (ArF) imaging radiation. The methods of the invention preferably comprise:
(a) providing a material surface on a substrate,
(b) forming a photoresist layer over the material surface, the photoresist being a photoresist of the invention,
(c) patternwise exposing said photoresist layer to radiation thereby creating a pattern of radiation-exposed regions in said photoresist layer,
(d) selectively removing portions of said photoresist layer to expose portions of said material surface, and
(e) etching or ion implanting said exposed portions of said material, thereby forming said patterned material feature.

The material surface of the substrate may be a metal conductor layer, a ceramic insulator layer, a semiconductor layer or other material depending on the stage of the manufacture process and the desired material set for the end product. The compositions of the invention are especially useful for lithographic processes used in the manufacture of integrated circuits on semiconductor substrates. The compositions of the invention in lithographic processes to create patterned material layer structures such as metal wiring lines, holes for contacts or vias, insulation sections (e.g., damascene trenches or shallow trench isolation), trenches for capacitor structures, ion implanted semiconductor structures for transistors, etc. as might be used in integrated circuit devices.

In some cases, a bottom antireflective coating and/or underlayer coating (e.g., a planarizing underlayer) may be applied between the photoresist layer and the material surface. In some cases, a top antireflective coating layer may be applied over the photoresist layer (i.e., on the side of the photoresist layer distal from the material surface). The invention is not limited to the use of antireflective reflective coatings and/or underlayer materials, nor specific compositions of those coatings or materials.

The photoresist layer is then patternwise-exposed to the desired radiation (e.g. 193 nm ultraviolet radiation). The patternwise exposure is conducted through a mask which is placed over the photoresist layer. For 193 nm UV radiation, the total exposure energy is preferably about 100 millijoules/cm² or less, more preferably about 50 millijoules/cm² or less (e.g. 15-30 millijoules/cm²).

After the desired patternwise exposure, the photoresist layer is typically baked to further complete the acid-catalyzed reaction and to enhance the contrast of the exposed pattern. The post-exposure bake is preferably conducted at about 60-175° C., more preferably about 90-160° C. The post-exposure bake is preferably conducted for about 30 seconds to 5 minutes.

After post-exposure bake, if any, the photoresist structure with the desired pattern is obtained (developed) by contacting the photoresist layer with an aqueous alkaline solution which selectively dissolves the areas of the photoresist which were exposed to radiation in the case of a positive photoresist (or the unexposed areas in the case of a negative photoresist). Preferred aqueous alkaline solutions (developers) are aqueous solutions of tetramethyl ammonium hydroxide. The resulting lithographic structure on the substrate is then typically dried to remove any remaining developer. If a top antireflective coating has been used, it is preferably also dissolved by the developer in this step.

The pattern from the photoresist structure may then be transferred to the exposed portions of underlying material of the substrate by etching with a suitable etchant using techniques known in the art; preferably the transfer is done by reactive ion etching or by wet etching. Once the desired pattern transfer has taken place, any remaining photoresist may be removed using conventional stripping techniques. Alternatively, the pattern may be transferred by ion implantation to form a pattern of ion implanted material.

Examples of general lithographic processes where the composition of the invention may be useful are disclosed in U.S. Pat. Nos. 4,855,017; 5,362,663; 5,429,710; 5,562,801; 5,618,751; 5,744,376; 5,801,094; 5,821,469 and 5,948,570. Other examples of pattern transfer processes are described in Chapters 12 and 13 of “Semiconductor Lithography, Principles, Practices, and Materials” by Wayne Moreau, Plenum Press, (1988). It should be understood that the invention is not limited to any specific lithography technique or device structure.

The invention is further described by the examples below. The invention is not limited to the specific details of the examples.

### EXAMPLES

### Example 1

### Synthesis of Silver 4-cyanobenzenesulfonate

To a solution of 4-cyanobenzenesulfonyl chloride (3 g, 14.88 mmol) in 50 mL of acetonitrile and 2 mL of water was added silver carbonate (4.1 g, 14.88 mmol) in portions in darkness. The resulting suspension was stirred overnight for 4 days, until no starting material is shown on the thin layer chromatography with an eluant of hexane/ethyl acetate (1:4). The mixture was filtered through half an inch of Celite® and the solid was washed with 3×50 mL acetonitrile. The organic filtrate was combined and organic solvent was removed via rotary evaporator to dryness and thus afforded 4.3 g of white solid with a yield of 99.6%. The resulting compound was not purified for further reactions. ¹HNMR, d6-DMSO: 7.81 (d, 2H, 8.8 Hz), 7.75 (d, 2H, 8.4 Hz). ¹³CNMR, d6-DMSO: 152.60, 132.62, 126.91, 119.10, 111.73.

### Example 2

### Synthesis of Triphenyl Sulfonium 4-cyanobenzenesulfonate. (TPSCN)

To a solution of silver 4-cyanobenzenesulfonate (1.16 g, 4 mmol) in 50 mL of acetonitrile and 2 mL of water was added a solution of triphenyl sulfonium bromide (1.373 g, 4 mmol) in 20 mL of acetonitrile and 5 mL of water. The resulting mixture was stirred overnight for 3 days and the solid was allowed to precipitate for 1 day before it was filtered. The organic solvent was removed via rotary evaporator and the residue was re-dissolved in 60 mL of 2-butanone. The resulting solution was washed with 3×10 mL of water, dried over magnesium sulfate and filtered though Celite® and aluminum oxide basic. The organic solvent wad removed via rotary evaporator and dried over vacuum oven to dryness and thus afforded 0.97 g of product with a yield of 55%. ¹HNMR, d6-DMSO: 7.90-7.74 (m, 19H). ¹³CNMR, d6-DMSO: 153.11, 134.83, 132.54, 131.84, 131.74, 126.89, 125.62, 119.15, 111.50. DSC (10° C./min, nitrogen 5 mL/min) showed no obvious decomposition up to 250° C.

### Example 3

### Synthesis of Tert-butylphenyldiphenyl Sulfonium 4-cyanobenzenesulfonate. (DPTBPSCN)

To a solution of silver 4-cyanobenzenesulfonate (1.16 g, 4 mmol) in 50 mL of acetonitrile and 2 mL of water was added a solution of tert-butylphenyldiphenyl sulfonium bromide (1.6 g, 4 mmol) in 20 mL of acetonitrile and 5 mL of water. The resulting mixture was stirred overnight for 3 days and the solid was allowed to precipitate for 1 day before it was filtered. The organic solvent was removed via rotary evaporator and the residue was re-dissolved in 50 mL of 2-butanone. The resulting solution was washed with 2×15 mL of water, dried over magnesium sulfate and filtered though Celite® and aluminum oxide basic. The organic solvent wad removed via rotary evaporator and dried over vacuum oven to dryness and thus afforded 1.55 g of product with a yield of 77%. ¹HNMR, d6-DMSO: 7.89-7.73 (m, 18H), 1.32 (s, 9H). ¹³CNMR, d6-DMSO: 158.24, 153.12, 134.74, 132.53, 131.81, 131.71, 131.58, 128.89, 126.89, 125.90, 122.10, 119.15, 111.49, 35.63, 31.09. DSC (10° C./min, nitrogen 5 mL/min) showed no obvious decomposition up to 250° C.

### Example 4

### Synthesis of Di(tert-butylphenyl)iodonium 4-cyanobenzenesulfonate. (DTBPICN)

To a solution of silver 4-cyanobenzenesulfonate (0.87 g, 3 mmol) in 40 mL of acetonitrile was added a solution of di(tert-butylphenyl) iodonium acetate (1.36 g, 3 mmol) in 20 mL of acetonitrile and 5 mL of water. The resulting mixture was stirred overnight for 5 days before it was filtered through Celite® and the remaining solid was washed by 50 mL of acetonitrile. The organic solvent was then removed via rotary evaporator and the residue was taken by 200 mL of 2-butanone. The insoluble solid was removed by filtration and the filtrate was concentrated to dryness via rotary evaporator, re-dissolved in 160 mL of 2-butanone and washed by 2×40 mL of water. The resulting organic layer was then dried over magnesium sulfate, filtered though Celite® and basic aluminum oxide, concentrated via rotary evaporator, dried over vacuum oven to dryness and thus afforded 1.2 g of product with a yield of 70%. ¹HNMR, d6-DMSO: 8.15 (d, 8.4 Hz, 4H), 7.80 (d, 8.4 Hz, 2H), 7.75 (d, 8.4 Hz, 2H), 7.54 (d, 8.8 Hz, 4H), 1.26 (s, 18H). DSC (10° C./min, nitrogen 5 mL/min): Td, 196° C.

### Example 5

### Synthesis of 1-(2-oxo-2-p-tolyl-ethyl)-tetrahydro-thiophenium Bromide

To a solution of 2-bromo-4′-methylacetophenone (19.18 g, 90 mmol) in 120 mL of acetone and 4.9 mL of water at 0° C. was added tetrahydrothiophene (15.87 g, 180 mmol) dropwise. The resulting mixture was stirred at 0° C. for 1 hour before solid precipitated and was then stirred at room temperature for 1 hour. The resulting crude product was filtered through glass frit as solid and was washed by 100 mL of cold acetone. The crude product was recrystallized by 150 mL of ethanol to afford 19.8 g pure product at a yield of 73%. ¹HNMR, d6-DMSO: 7.92 (d, 8.0 Hz, 2H), 7.43 (d, 8.0 Hz, 2H), 5.39 (s, 2H), 3.57 (m, 4H), 2.42 (s, 3H), 2.27 (m, 2H), 2.19 (m, 2H).

### Example 6

### Synthesis of 1-(2-oxo-2-p-tolyl-ethyl)-tetrahydro-thiophenium 4-cyanobenzenesulfonate (BCN)

To a solution of silver 4-cyanobenzenesulfonate (0.87 g, 3 mmol) in 55 mL of acetonitrile and 2 mL of water was added a solution of 1-(2-oxo-2-p-tolyl-ethyl)-tetrahydro-thiophenium bromide (0.904 g, 3 mmol) in 30 mL of acetonitrile and 3 mL of water. The resulting mixture was stirred overnight for 3 days before it was filtered through Celite® and the remaining solid was washed by 50 mL of acetonitrile. The organic solvent was then removed via rotary evaporator and the residue was taken by 100 mL of 2-butanone. and the solution was washed by 2×10 mL of water. The resulting organic layer was then dried over magnesium sulfate, filtered though Celite® and basic aluminum oxide, concentrated via rotary evaporator, dried over vacuum oven to dryness and thus afforded 0.97 g of product at a yield of 80%. ¹HNMR, d6-DMSO: 7.91 (d, 8.4 Hz, 2H), 7.79 (d, 8.4 Hz, 2H), 7.75 (d, 8.4 Hz, 2H), 7.44 (d, 8.0 Hz, 2H), 3.59 (m, 2H), 3.52 (m, 2H), 2.42 (s, 3H), 2.32-2.12 (brm, 4H), DSC (10° C./min, nitrogen 5 mL/min): Td, 237° C.

### Example 7

### Synthesis of 1-(2-naphthalen-2-yl-2-oxo-ethyl)-tetrahydro-thiophenium Bromide

To a solution of 2-bromo-2′-acetonaphthone (18.68 g, 75 mmol) in 100 mL of acetone and 4.1 mL of water at 0° C. was added tetrahydrothiophene (13.23 g, 150 mmol) dropwise. The resulting mixture was stirred at 0° C. for 1 hour before solid precipitated and was then stirred at room temperature for 1 hour. The resulting crude product was filtered through glass frit as solid and was washed by 100 mL of cold acetone. The crude product was recrystallized by 150 mL of ethanol to afford 19.0 g pure product at a yield of 75%. ¹HNMR, d6-DMSO: 8.82 (s, 1H), 8.17-7.98 (m, 4H), 7.78-7.65 (m, 2H), 5.63 (s, 2H), 3.64 (m, 4H), 2.40-2.29 (brm, 2h), 2.28-2.17 (brm, 2H).

### Example 8

### Synthesis of 1-(2-naphthalen-2-yl-2-oxo-ethyl)-tetrahydro-thiophenium 4-cyanobenzenesulfonate (NCN)

To a solution of silver 4-cyanobenzenesulfonate (0.87 g, 3 mmol) in 50 mL of acetonitrile and 3 mL of water was added a solution of 1-(2-naphthalen-2-yl-2-oxo-ethyl)-tetrahydro-thiophenium bromide (1.012 g, 3 mmol) in 35 mL of acetonitrile and 3 mL of water. The resulting mixture was stirred overnight for 3 days before it was filtered through Celite® and the remaining solid was washed by 50 mL of acetonitrile. The organic solvent was then removed via rotary evaporator and the residue was taken by 100 mL of 2-butanone. The resulting organic solution was then dried over magnesium sulfate, filtered though Celite® and basic aluminum oxide, concentrated via rotary evaporator, dried over vacuum oven to dryness and thus afforded 1.12 g of product at a yield of 85%. ¹HNMR, d6-DMSO: 8.74 (s, 1H), 8.18-7.98 (m, 4H), 7.8 (d, 8.4 Hz, 2H), 7.75 (d, 8.4 Hz, 2H), 7.79-7.67 (brm, 2H), 5.47 (s, 2H), 3.68-3.52 (m, 4H), 2.36-2.15 (m, 4H), DSC (10° C./min, nitrogen 5 mL/min): Td, 220° C.

Comparison Photoacid Generators

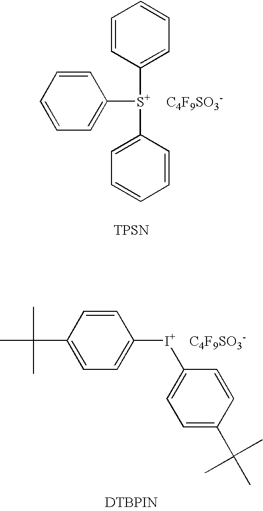

### Example 9

### Photoresist Formulation 1 (Comparison Example)

5.2959 g of a photoresist polymer consisting of 15 mol % of 2-trifluoromethanesulfonylamino methacrylate, 45 mol % of 2-methyl-2-adamantyl methacrylate and 40 mol % of 5-methacryloyloxy-2,6-norbornanecarbo-γ-lactone (S1) (28.3 wt % solution in PGMEA), 0.3739 g of triphenyl sulfonium nonafluorobutanesulfonate (TPSN) (20.28 wt % solution in PGMEA), 0.4528 g of N-t-Boc-pyrrolidine (1 wt % solution in PGMEA), 10.1472 g of PGMEA and 6.2863 g of cyclohexanone were mixed and rotated overnight, filtered though 0.2 μm PTFE disc to afford formulation 1.

### Example 10

### Photoresist Formulation 2

5.3089 g of photoresist polymer S1 (28.3 wt % solution in PGMEA), 0.0589 g of TPSCN, 0.4544 g of N-t-Boc-pyrrolidine (1 wt % solution in PGMEA), 10.2952 g of PGMEA and 6.2295 g of cyclohexanone were mixed and rotated overnight, filtered though 0.2 μm PTFE disc to afford formulation 2.

### Example 11

### Photoresist Formulation 3

5.3034 g of photoresist polymer S1 (28.3 wt % solution in PGMEA), 0.0671 g of DPTBPSCN, 0.4608 g of N-t-Boc-pyrrolidine (1 wt % solution in PGMEA), 10.3621 g of PGMEA and 6.2831 g of cyclohexanone were mixed and rotated overnight, filtered though 0.2 μm PTFE disc to afford formulation 3.

### Example 12

### Photoresist Formulation 4

5.3107 g of photoresist polymer S1 (28.3 wt % solution in PGMEA), 0.0768 g of DTBPICN, 0.4588 g of N-t-Boc-pyrrolidine (1 wt % solution in PGMEA), 10.4517 g of PGMEA and 6.3038 g of cyclohexanone were mixed and rotated overnight, filtered though 0.2 μm PTFE disc to afford formulation 4.

### Example 13

### Photoresist Formulation 5 (Comparison Example)

5.3093 g of photoresist polymer S1 (28.3 wt % solution in PGMEA), 0.0926 g of DTBPIN, 0.4577 g of N-t-Boc-pyrrolidine (1 wt % solution in PGMEA), 10.6040 g of PGMEA and 6.3728 g of cyclohexanone were mixed and rotated overnight, filtered though 0.2 μm PTFE disc to afford formulation 5

### Example 14

### Photoresist Formulation 6

5.3003 g of photoresist polymer S1 (28.3 wt % solution in PGMEA), 0.0544 g of BCN, 0.4548 g of N-t-Boc-pyrrolidine (1 wt % solution in PGMEA), 10.2419 g of PGMEA and 6.2844 g of cyclohexanone were mixed and rotated overnight, filtered though 0.2 μm PTFE disc to afford formulation 6.

### Example 15

### Photoresist Formulation 7

5.3001 g of photoresist polymer S1 (28.3 wt % solution in PGMEA), 0.0591 g of NCN, 0.4552 g of N-t-Boc-pyrrolidine (1 wt % solution in PGMEA), 10.2836 g of PGMEA and 6.2319 g of cyclohexanone were mixed and rotated overnight, filtered though 0.2 μm PTFE disc to afford formulation 7.

### Example 16

### Photoresist Formulation 8

0.7524 g of a photoresist polymer consisting 60 mol % of 2-methyl-acrylic acid-(3,3,3-trifluoro-2-hydroxy-2-trilfluoromethyl-propyl)-bicyclo[2,2,1]hept-2-yl-ester and 40 mol % of 2-methyl-2-adamantyl methacrylate (polymer F1), 0.0149 g of TPSCN, 0.1205 g of N-t-Boc-pyrrolidine (1 wt % solution in PGMEA), 7.4717 g of PGMEA and 2.6461 g of cyclohexanone were mixed and rotated overnight, filtered though 0.2 μm PTFE disc to afford formulation 8.

### Physical Properties

Thin solid films were prepared by spin-coating photoresist formulations over 5 inch silicon wafers at the spin rate of 1500 rpm for 30 seconds. The resulting films were soft baked in vacuum at 110° C. for 60 seconds. The thickness, n and k were measured by VASE ellipsometry, OD values were calculated from k. (Table 1)

For lithographic evaluation, the prepared photoresist formulation was spin-coated for 30 seconds onto an antireflective coating material (AR 40, Rohm-Haas) layer applied on silicon wafers. The photoresist films were baked at 110° C. on a vacuum hotplate for 60 seconds. The wafers were then exposed to ArF radiation (ASML, scanner, 0.75 NA). The exposure pattern was an array of lines and spaces of various dimensions down to 90 nm. The exposed wafer was then post-exposure baked on a vacuum hot plate at 120° C. for 90 seconds. The wafers were puddle developed by 0.263 N TMAH developer for 60 seconds. The resulting patterns of the photoresist imaging layers were examined by scanning electron microscopy (SEM). The Photospeed results were obtained the images of 90 nm line in 245 nm pitch.

### Leaching Tests:

Photoresist formulation 1 was spin-coated on bare silicon wafer at 1500 rpm for 30 seconds and baked at 110° C. on a vacuum hotplate for 60 seconds. Topcoat TCX-041 was spin-coated onto the resulting photoresist film at 1500 rpm for 30 seconds and soft baked on a vacuum hotplate at 90° C. for 60 seconds. The resulting wafer was evaluated by water leaching test. 0.275 ng/mL of PAG TPSCN in water leaching sample was found by HPLC-MS test.

TABLE 1
Physical properties and lithography performance of the photoresist
composite thin films containing aromatic PAGs.

| | | Thickness | n | | OD | Photospeed |
|---|---|---|---|---|---|---|
| Formulation | PAG | (nm) | (193 nm) | k | (μm⁻¹) | (mJ) |
| 1 | TPSN | 205.64 | 1.6993 | 0.034978 | 0.99 | 14.5 |
| 2 | TPSCN | 209.85 | 1.6981 | 0.039430 | 1.11 | 19.25 |
| 3 | DPTBPSCN | 210.31 | 1.7014 | 0.038905 | 1.10 | 26.75 |
| 4 | DTBPICN | 210.31 | 1.6994 | 0.042869 | 1.21 | 56 |
| 5 | DPTBPIN | 215.15 | 1.7007 | 0.030923 | 0.87 | 18.5 |
| 6 | BCN | 222.42 | 1.6998 | 0.018390 | 0.52 | |
| 7 | NCN | 218.57 | 1.6987 | 0.021442 | 0.61 | |

1-4. (canceled)

5. A chemically amplified photoresist composition suitable for use with imagine radiation from an ArF laser said composition comprising:
(a) an acid sensitive imaging polymer having at least one lactone moiety, and
(b) a fluorine-free photoacid generator comprising an anionic component having the structure 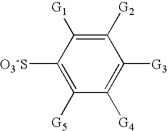
wherein at least one of G₁-G₅ is an electron withdrawing moiety selected from the group consisting of CN, NO, NO₂, Cl. Br, I, SO₂Me, CHO;
said photoresist composition being free of fluorine-containing acid generators.

6. (canceled)

7. The composition of claim 5 wherein the G₁-G₅ moieties that are not electron withdrawing moieties are selected independently from the group consisting of H; linear, branched, tertiary, or cyclic alkyl; linear, branched, tertiary or cyclic alkoxyl; unsubstituted and substituted phenyl; unsubstituted and substituted naphthyl; or unsubstituted and substituted fluorenyl.

8. The composition of claim 5 wherein at least two of G₁-G₅ are electron withdrawing moieties.

9. The composition of claim 5 wherein said photoacid generator further comprises an onium cationic component.

10. The composition of claim 9 wherein said onium cationic component is selected from the group consisting of sulfonium and iodonium.

11-14. (canceled)

15. A method of forming a patterned material feature on a substrate, said method comprising:
(a) providing a material surface on a substrate,
(b) forming a photoresist layer over said material surface, said photoresist comprising
(i) an acid sensitive imaging polymer having at least one lactone moiety, and
(ii) a fluorine-free photoacid generator comprising an anionic component having the structure 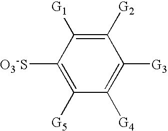
wherein at least one of G₁-G₅ is an electron withdrawing moiety selected from the group consisting of CN, NO, NO₂, Cl, Br, I, SO₂Me, CHO, said photoresist composition being free of fluorine-containing acid generators.
(c) patternwise exposing said photoresist layer to radiation provided by an ArF laser thereby creating a pattern of radiation-exposed regions in said photoresist layer,
(e) selectively removing portions of said photoresist layer to expose portions of said material surface, and
(f) etching or ion implanting said exposed portions of said material, thereby forming said patterned material feature.

16-19. (canceled)

20. The composition of claim 5 wherein electron withdrawing moiety is CN.

21. The method of claim 15 wherein electron withdrawing moiety is CN.
